(19) United States
(12) Patent Application Publication
(10) Pub. No.: US 2026/0045351 A1
(43) Pub. Date: Feb. 12, 2026

(54) MONITORING USAGE OF A HEMORRHOID TREATMENT DEVICE AT A SURGICAL CENTER

(71) Applicant: MEDFINITI IP, LLC, Las Vegas, NV (US)

(72) Inventors: Stephen Goldman, Palm Harbor, FL (US); Michael J. Goeree, Tampa (CA)

(21) Appl. No.: 19/174,481

(22) Filed: Apr. 9, 2025

Related U.S. Application Data
(63) Continuation-in-part of application No. 18/795,908, filed on Aug. 6, 2024.

(51) Int. Cl. G16H 20/40; A61B 18/12; G16H 15/00

(52) U.S. Cl. CPC G16H 20/40; A61B 18/12; G16H 15/00

(57) ABSTRACT

A method for monitoring usage of a hemorrhoid treatment device includes registering with a backend server the hemorrhoid treatment device provided to a patient treatment center, and registering with the backend server a physician at the patient treatment center to operate the hemorrhoid treatment device. The hemorrhoid treatment device is coupled to a computing device at the patient treatment center and includes operational treatment software configured to communicate with the backend server. The physician logs into the operational treatment software on the computing device. The hemorrhoid treatment device is operated by the physician to treat a patient with hemorrhoids. Usage of the hemorrhoid treatment device by the physician is monitored via the operational treatment software. A treatment summary report is generated via the operational treatment software after the patient with hemorrhoids has been treated.

### RELATED APPLICATION

The present application is a continuation-in-part of U.S. patent application Ser. No. 18/795,908, filed on Aug. 6, 2024, which is incorporated herein by reference in its entirety.

### TECHNICAL FIELD

The present disclosure relates to the treatment of hemorrhoids, and, more particularly, to monitoring usage of a hemorrhoid treatment device.

### BACKGROUND

Hemorrhoids are part of the normal rectal anatomy and consist of arteries/arterioles, columns and venules/veins. Columns are sinus-like structures without smooth muscle. Columns are a unique anatomical structure of the rectum. In all other tissues arteries/arterioles are connected to venules/veins by capillaries. The hemorrhoid consists of three components including arteries, columns and veins. The hemorrhoid may enlarge and protrude into the rectum or externally the rectum. When this occurs, it becomes pathological and is referred to as a hemorrhoid disease. Hemorrhoids are generally classified as being either internal or external, depending on their location relative to the dentate line.

The pain often associated with hemorrhoids may be great enough to interfere with normal patterns of defecation which in turn leads to constipation and further aggravation of the swelling in the region. In general, most symptoms associated with hemorrhoids are sufficient enough to interfere with a person's daily life causing them to require relief from a medical practitioner and/or a pharmaceutical preparation.

There is a wide range of therapies available that attempt to provide a person suffering from hemorrhoids with some level of relief. One option is medicated ointments and creams which generally relieve either the itching or inflammation, but few have been successful in reducing or completely eliminating both. Thus, the efficacy of medicated ointments and creams in relieving or curing the symptoms of hemorrhoids is uncertain.

Another option for treating hemorrhoids is surgery. Treatment via surgery ranges from minor surgery to major invasive surgery. Minor surgery may relieve symptoms through drainage of the swelling. Major invasive surgery, for example, includes the removal of extensive or severe hemorrhoids in a process known as a hemorrhoidectomy. This surgical procedure can be used on both internal and external hemorrhoids. However, a hemorrhoidectomy typically involves a long recovery period, along with the associated risks and expense of invasive surgery.

Another example for treating hemorrhoids is to deliver an electrical current to the hemorrhoids. U.S. Pat. No. 9,179,966 provides a method of treating hemorrhoids that includes inputting a predetermined current time unit into a current delivering medical instrument, wherein the predetermined current time unit is based on the grade of the hemorrhoid. The hemorrhoid is contacted with a probe electrode of the current delivering medical instrument, and current is delivered to the hemorrhoid. A delivered current time unit is determined. The delivered current time unit includes a product comprised of current delivered multiplied by time. A visual and/or auditory indication is provided with the medical instrument when the predetermined current time unit amount is achieved.

### SUMMARY

A method for monitoring usage of a hemorrhoid treatment device includes registering with a backend server the hemorrhoid treatment device provided to a patient treatment center, and registering with the backend server a physician at the patient treatment center to operate the hemorrhoid treatment device. The hemorrhoid treatment device is coupled to a computing device at the patient treatment center, with the computing device including operational treatment software configured to communicate with the backend server.

The method includes the physician logging into the operational treatment software on the computing device, with the operational treatment software verifying with the backend server that the physician is registered to operate the hemorrhoid treatment device. The hemorrhoid treatment device is operated by the physician to treat a patient with hemorrhoids. The method includes monitoring usage of the hemorrhoid treatment device by the physician via the operational treatment software, and generating a treatment summary report via the operational treatment software after the patient with hemorrhoids has been treated.

The method includes providing the treatment summary report to the backend server. The backend server generates an invoice for the patient treatment center in response to receiving the patient treatment report. The backend server then sends the invoice to the patient treatment center.

The method includes performing a self-test on the hemorrhoid treatment device before the physician logs into the operational treatment software.

The treatment summary report includes identification of the patient, how many hemorrhoids were treated, and how long current was delivered to the hemorrhoids by the hemorrhoid treatment device.

Operating the hemorrhoid treatment device includes delivering an output voltage to a probe electrode coupled to the hemorrhoid treatment device, with the probe electrode to contact the hemorrhoid being treated, and adjusting the output voltage to compensate for fluctuations in body resistance of the patient so that a level of current being delivered to the hemorrhoid matches a set level of current.

The hemorrhoid treatment device includes a voltage output configured to deliver the output voltage to the probe electrode, and a variable resistance network coupled to the voltage output. Operating the hemorrhoid treatment device includes changing resistance of the variable resistance network to adjust the output voltage to compensate for the fluctuations in body resistance of the patient so that the set level of current being delivered to the hemorrhoid is consistent.

The method includes positioning a grounding pad to contact the patient, with the grounding pad being coupled to the hemorrhoid treatment device to be used in determining the fluctuations in body resistance of the patient.

Another aspect is directed to a hemorrhoid treatment system that includes a backend server, and a computing device at a patient treatment center. The computing device includes operational treatment software in communications with the backend server. A hemorrhoid treatment is coupled to the computing device. The hemorrhoid treatment device is registered with the backend server. The hemorrhoid treatment device is to be operated by a physician at the patient treatment center after logging into the operational treatment software, with the physician being registered with the backend server to operate the hemorrhoid treatment device. The operational treatment software is configured to monitor usage of the hemorrhoid treatment device by the physician to treat a patient with hemorrhoids via the operational treatment software, and generate a treatment summary report via the operational treatment software after the patient with hemorrhoids has been treated.

### BRIEF DESCRIPTION OF THE DRAWINGS

FIG. 1 is a block diagram of a hemorrhoid treatment system in which various aspects of the disclosure may be implemented.

FIG. 2 is a view of the handheld device, probe electrode, grounding pad and grounding pad cable illustrated in FIG. 1.

FIG. 3 is a back side perspective view of the handheld device and probe electrode illustrated in FIG. 1 without the cable.

FIG. 4 is a front side perspective view of the handheld device and probe electrode illustrated in FIG. 1 without the cable.

FIG. 5A is a side view of a single length of electrically conductive material bent in half to form part of the probe electrode illustrated in FIG. 1.

FIG. 5B is a side view of the electrically conductive material illustrated in FIG. 5A with shrink tubing.

FIG. 5C is a back side perspective view of the electrically conductive material with shrink tubing illustrated in FIG. 5B with a base overmold.

FIG. 6A is a back side perspective view of the probe electrode illustrated in FIG. 5C with a slidable spacer.

FIG. 6B is a front view of the slidable spacer illustrated in FIG. 6A.

FIGS. 7A-7C are different views of another embodiment of the probe electrode illustrated in FIG. 5C with a base overmold closed extension.

FIGS. BA-8C are different views of another embodiment of the probe electrode illustrated in FIG. 5C with a base overmold open extension.

FIG. 9 is a partially exploded view of the handheld device configured to receive the probe electrode illustrated in FIG. 1.

FIG. 10 is a partial view of the probe electrode coupled to the handheld device illustrated in FIG. 9.

FIGS. 11A-11C are sequence views of the closed end of the probe electrode being inserted into the electrical plug within the handheld device illustrated in FIG. 10.

FIG. 12 is a view of a user interface membrane placed on the user interface area of the handheld device illustrated in FIG. 3.

FIG. 13 is a flowchart for using the handheld device illustrated in FIG. 1 to treat a hemorrhoid on a patient.

FIG. 14 is a block diagram of a hemorrhoid treatment system in which usage of the hemorrhoid treatment device is monitored at a patient treatment center.

FIG. 15 is a screenshot displayed by the computing device illustrated in FIG. 14 prompting self-test of the hemorrhoid treatment device.

FIG. 16 is a screenshot displayed by the computing device illustrated in FIG. 14 providing a login page for the physician.

FIG. 17 is a screenshot displayed by the computing device illustrated in FIG. 14 allowing the physician to perform a new procedure.

FIG. 18 is a screenshot displayed by the computing device illustrated in FIG. 14 allowing the physician to enter information on a patient to be treated with hemorrhoids.

FIGS. 19-22 are screenshots displayed by the computing device illustrated in FIG. 14 tracking usage of the hemorrhoid treatment device by the physician.

FIG. 23 is a screenshot displayed by the computing device illustrated in FIG. 14 prompting the physician to confirm logging out of the operational treatment software.

FIG. 24 is a screenshot displayed by the computing device illustrated in FIG. 14 providing a treatment summary report.

FIG. 25 is a flowchart for monitoring usage of the hemorrhoid treatment device illustrated in FIG. 14.

FIG. 26 is a flowchart for operating the hemorrhoid treatment device illustrated in FIG. 14.

### DETAILED DESCRIPTION

The present description is made with reference to the accompanying drawings, in which exemplary embodiments are shown. However, many different embodiments may be used, and thus the description should not be construed as limited to the particular embodiments set forth herein. Rather, these embodiments are provided so that this disclosure will be thorough and complete. Like numbers refer to like elements throughout.

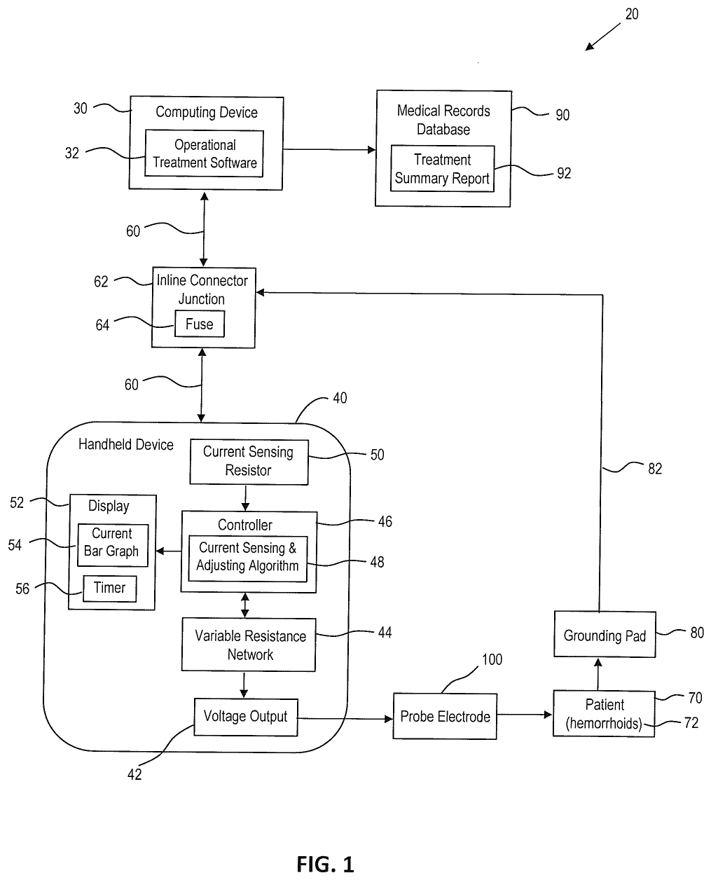

Referring initially to FIG. 1, a hemorrhoid treatment system 20 for a patient 70 having hemorrhoids 72 that are to be treated will be discussed. The hemorrhoid treatment system 20 utilizes direct current electrotherapy to reduce and eliminate symptoms of hemorrhoid disease, which is estimated to affect up to one-third of the population in the United States.

As will be described in detail below, the hemorrhoid treatment system 20 is advantageously configured to automatically deliver a steady level of current to the hemorrhoid 72 by using feedback to adjust an output voltage to compensate for fluctuations in body resistance of the patient 70. As noted in the above Background section, U.S. Pat. No. 9,179,966 disclosed the use of electrical current to treat hemorrhoids. A limitation of the '966 patent is that manual intervention was required to adjust for variations in the level of current being delivered to the hemorrhoid due to fluctuations in body resistance of the patient.

The hemorrhoid treatment system 20 includes a computing device 30, and a handheld device 40 coupled to the computing device 30 via a cable 60. The computing device 30 is configured to provide power and ground to the handheld device 40 over the cable 60, as well as to exchange data signals with the handheld device 40.

The computing device 30 may be a laptop or desktop computing device, for example. The computing device 30 is configured to execute operational treatment software 32 for interfacing with the handheld device 40 over the cable 60. One of the functions of the operational treatment software 32 is to perform handshaking with the handheld device 40. Handshaking is required to authorize operation of the handheld device 40. After authorization, operation of the handheld device 40 may be self-sustained to operate on its own.

The operational treatment software 32 is also configured to generate a treatment summary report 92 after the patient 70 has been treated, which may be used for billing purposes. The treatment summary report 92 may include identification of the patient, a duration of how long current was delivered to the hemorrhoid 72, and the level of the current delivered to the hemorrhoid 72 during the duration. The patient identification may be, for example, by name, social security number, or an assigned patient number. The treatment summary report 92 may be stored in a medical records database 90.

A probe electrode 100 has a first end coupled to the handheld device 40, and a second end configured to contact or engage the hemorrhoid 72 that is to be treated. A grounding pad 80 is to be coupled to the patient 70. The cable 60 includes an inline connector junction 62. A grounding pad cable 82 is coupled between the grounding pad 80 and the inline connector junction 62, where the inline connector junction 62 provides a ground to the grounding pad 80. The inline connector junction 62 may include a fuse 64 in series between the grounding pad 80 and a grounding wire within the cable 60 to limit a maximum amount of current that may be delivered to the hemorrhoid 72.

The handheld device 40 includes a voltage output 42 configured to deliver an output voltage to the probe electrode 100, a variable resistance network 44 coupled to the voltage output 42, and a current sensing resistor 50 configured to provide a feedback voltage. The variable resistance network 44 has a plurality of selectable resistance configurations.

A controller 46 is coupled to the current sensing resistor 50 and to the variable resistance network 44. The controller 46 is configured to operate a current sensing and adjusting algorithm 48 to periodically sense the feedback voltage at the current sensing resistor 50 in response to the second end of the probe electrode 100 contacting the hemorrhoid 72, and to the patient 70 being grounded via the grounding pad 80. The patient 70 acts as a resistive load to form an electrical circuit between the probe electrode 100 and the grounding pad 80.

A level of current being delivered to the hemorrhoid 72 is determined by the current sensing and adjusting algorithm 48 using Ohm's law. Ohm's law is a formula used to calculate the relationship between voltage, current and resistance in an electrical circuit.

In response to the probe electrode 100 contacting the hemorrhoid 72 and the patient 70 being grounded via the grounding pad 80, an output voltage is applied to the probe electrode 100. The current flowing through the hemorrhoid 72 ramps up from 0 to 16 milliamps (mA) within about 10 seconds. The electrical current is a steady, low-level current that causes smooth muscle contraction and thrombosis resulting in permanent ligation of the hemorrhoid's feeder blood vessels. The use of 16 mA is one example current level that may be used for treating hemorrhoids 72. A 16 mA current may be applied for a time determined by the hemorrhoid's grade for a 90-95% cure rate. The doctor or gastroenterologist will determine the hemorrhoid grade and thus choose the procedural time needed. Since the vast majority of patients are unable to tolerate 16 mA of current, anesthesia is typically used.

As noted above, the level of current being delivered to the hemorrhoid 72 is determined by the controller 46 based on the feedback voltage being measured at the current sensing resistor 50. The feedback voltage is periodically measured. As an example, this measurement may be in the milliseconds range, such as every 32 milliseconds.

The handheld device 40 includes a display 52 coupled to the controller 46. The display 52 provides a current bar graph 54 and a timer 56. The current bar graph 54 displays the level of current being delivered to the hemorrhoid 72. When current is being delivered to the hemorrhoid 72, the timer 56 changes time. The handheld device 40 may operate in a manual mode or a timer mode. In the manual mode, the timer 56 counts up from zero. In the timer moder, the timer 56 counts down to zero from a time selected by the doctor.

In response to the level of current being delivered to the hemorrhoid 72, the controller 46 changes resistance of the variable resistance network 44 to adjust the output voltage to compensate for fluctuations in body resistance of the patient 70 so that the level of current being delivered to the hemorrhoid 72 matches a desired or set level of current. The desired level of current is 16 mA in this example embodiment, and may be considered a default value for the level of current to be applied to the hemorrhoid 72 without additional input from the doctor.

The controller 46 may have a plurality of output ports that interfaces with a corresponding plurality of inputs to the resistive network 44, where each input corresponds to a different resistance configuration. The controller 46 46 changes resistance of the variable resistance network 44 by selecting the appropriate output port that connects to a desired resistance configuration within the resistive network 44.

Fluctuations in body resistance, for example, may be due to movement by the patient 70 with respect to the grounding pad 80. In addition, saline is a conductive solution that is typically used to apply the grounding pad 80 to the patient 70. If the saline starts to dry out, then this may lead to fluctuations in body resistance of the patient 70. The handheld device 40 advantageously treats the hemorrhoid 72 by delivering the desired level of current to the hemorrhoid 72 within a very tight tolerance. The tolerance may be within a range of 0.5-1.5 percent, for example.

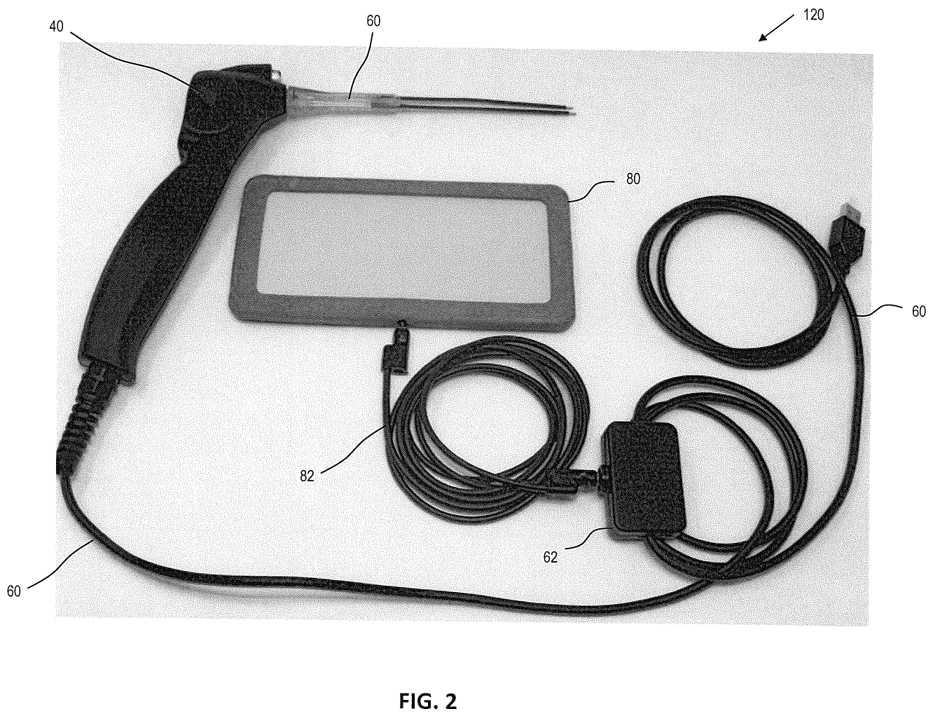

A view 120 of the handheld device 40 with cable 60 and inline connector junction 62, probe electrode 100, grounding pad 80 and grounding pad cable 82 is provided in FIG. 2. Since the grounding pad 80 is disposable, it is removably coupled to one end of the ground pad cable 82. The other end of the grounding pad cable 82 is removably coupled to the inline connector junction 62. One end of the cable 60 is coupled within the handheld device 40, with the free end of the cable 60 being removably coupled to the computing device 30. The cable 60 may be configured as a USB (universal serial bus) cable. A USB cable allows data exchanges between the computing device 30 and the handheld device 40, and provides power and ground to the handheld device 40 as well as providing ground to the ground pad 80 via the inline connector junction 62.

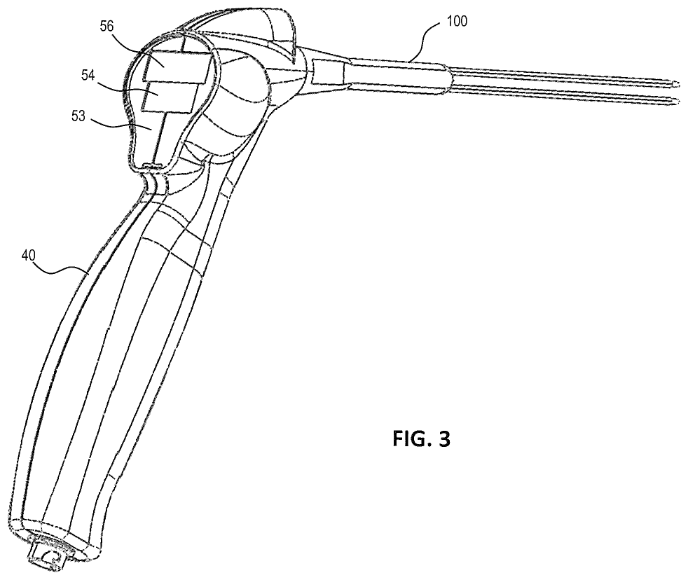

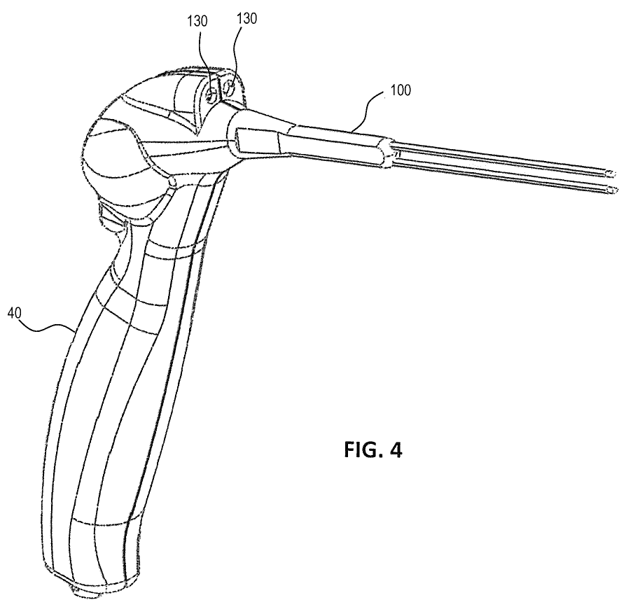

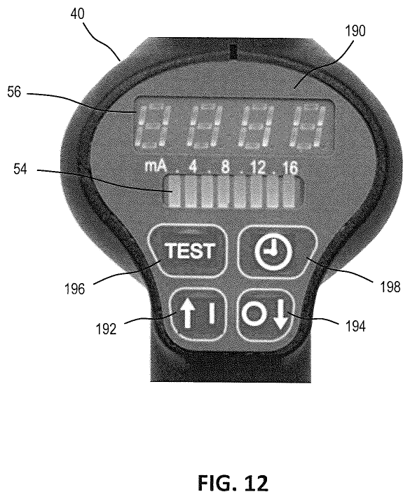

Referring now to FIGS. 3 and 4, a back side perspective view and a front perspective view of the handheld device 40 with the probe electrode 100 are provided. The handheld device 40 includes a user interface area 53 that is visible to the doctor from the backside of the handheld device 40. The display area 53 includes the current bar graph 54 and the timer 56. As will be discussed in greater detail below with reference to FIG. 12, a user interface membrane 190 is placed over the display area 53 which provides user interface buttons for operating the handheld device 40.

The handheld device 40 further includes at least one light 130 directed to the second end of the probe electrode 100 that contacts the hemorrhoid. This results in safer and more accurate procedures. In the illustrated embodiment, a pair of lights 130 are provided. The lights may be configured as light emitting diodes (LEDs).

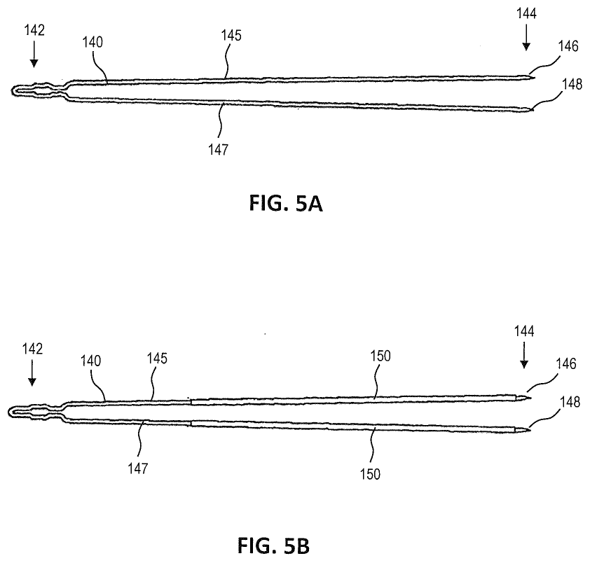

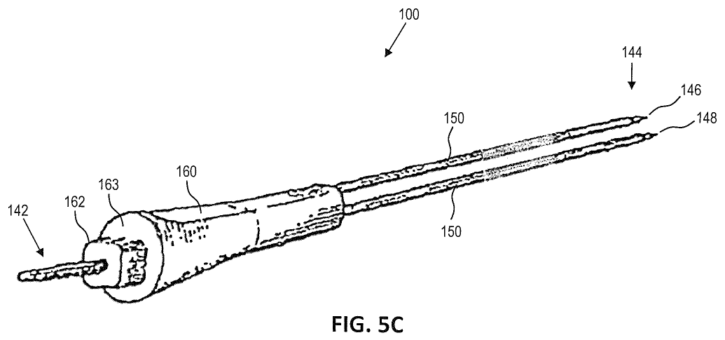

Referring now to FIGS. 5A-5C, the probe electrode 100 will be discussed in greater detail. The probe electrode 100 includes a single length of electrically conductive material 140 that is folded in half, with a bend region forming a closed end 142 of the probe electrode 100, as shown in FIG. 5A. Extending from the bend region is a first elongated electrode member 145 that includes a first electrode tip 146, and a second elongated electrode member 147 that includes a second electrode tip 146. The first and second electrode tips 146, 148 form an open end of the probe electrode 100. Each electrode tip 146, 148 is preferably beveled to form a point.

The closed end 142 corresponds to the first end of the probe electrode 100, and the open end 144 corresponds to the second end of the probe electrode 100. In other words, the probe electrode 100 is bifurcated as a pair of spaced apart elongated electrode members 145, 147 each with an electrode tip 146, 148 for contacting the hemorrhoid 72.

The electrically conductive material may be a metal wire, such as stainless steel, copper, etc. The metal wire is heat annealed at approximately its center point, and is then bent about 180 degrees to form the bend region. This allows for the electrode tips 146, 148 to be divergent from one another.

Shrink tubing 150 is placed over the first elongated electrode member 145 and over the second elongated electrode member 147 while leaving the respective electrode tips 146, 148 exposed, as shown in FIG. 5B. The single length of electrically conductive material 140 with the shrink tubing 150 thereon is placed into an oven at a temperature and time sufficient to shrink the tubing 150 around the respective first and second elongated electrode members 145, 147. Typically, 15 minutes @ 300-500 degrees F. is sufficient for this purpose.

After shrinking the tubing 150, a base overmold 160 is associated with the first and second elongated electrode members 145, 147, as shown in FIG. 5C. The base overmold 160 provides a grip area for holding the probe electrode 100 while interfacing (i.e., coupling/decoupling) with the handheld device 40. In one embodiment this is achieved through plastic mold injection. The molds are designed to seal around a portion of the first and second elongated electrode members 145, 147 including a portion of the shrink tubing 150.

In one embodiment, the plastic used for the base overmold 160 is C10 made by Adept Polymers, Ltd, and the injection is conducted @ 11, 000 PSI, @ 350-400 degrees F. The plastic injection forms a 4-sided keying block 162 with a physical stop 163. This is intended for mating with the handheld device 40. The 4-sided keying block 162 allows for the positioning of the probe electrode 100 in two vertical positions and two horizontal positions.

As noted above, the elongated electrode members 145, 147 and the electrode tips 146, 148 are divergent from one another. A default spacing of the electrode tips 146, 148 is obtained when the base overmold 160 is formed. For a default spacing of 5 mm, for example, the electrode tips 146, 148 are held at this spacing while the base overmold 160 is formed. After the base overmold 160 has been formed, the default spacing of the electrode tips 146, 148 is set. The default spacing of the electrode tips 146, 148 is not limited to 5 mm. The default spacing may vary within a range of 5 mm-12 mm, for example.

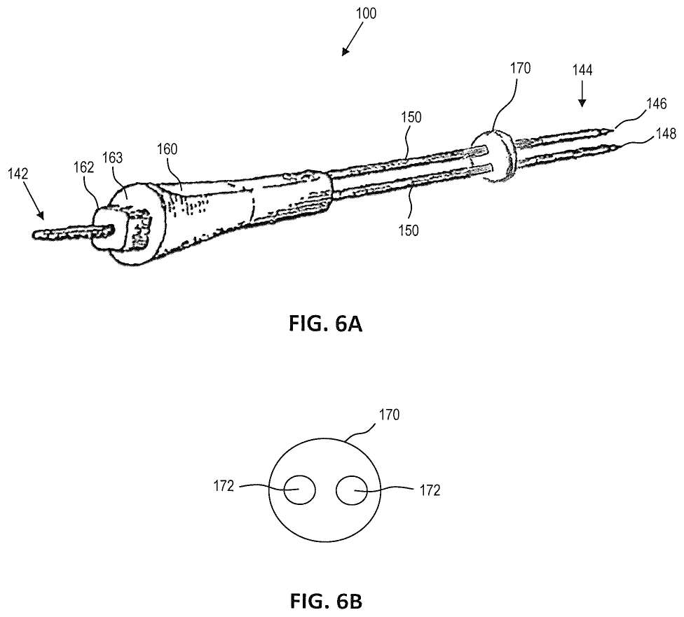

To ensure that spacing of the electrode tips 146, 148 is at the default range, or if the doctor would like to change the default spacing, a spacer 170 may be used, as shown in FIG. 6A. The spacer 170 includes a pair of spaced apart openings 172, as shown in FIG. 6B, for slidably engaging the elongated electrode members 145, 147 of the probe electrode 100.

The spacer 170 may be formed out of an elastic, flexible and moldable material, such as polyurethane or rubber, for example. Prior to treating the hemorrhoid 72, the doctor slides the spacer 170 over the electrode tips 146 148 and over the elongated electrode members 145, 147 until the desired spacing is achieved. The shrink tubing 150 placed over the elongated electrode members 145, 147 may include one or more markers calibrated to assist in positioning of the spacer 170 to achieve the desired spacing.

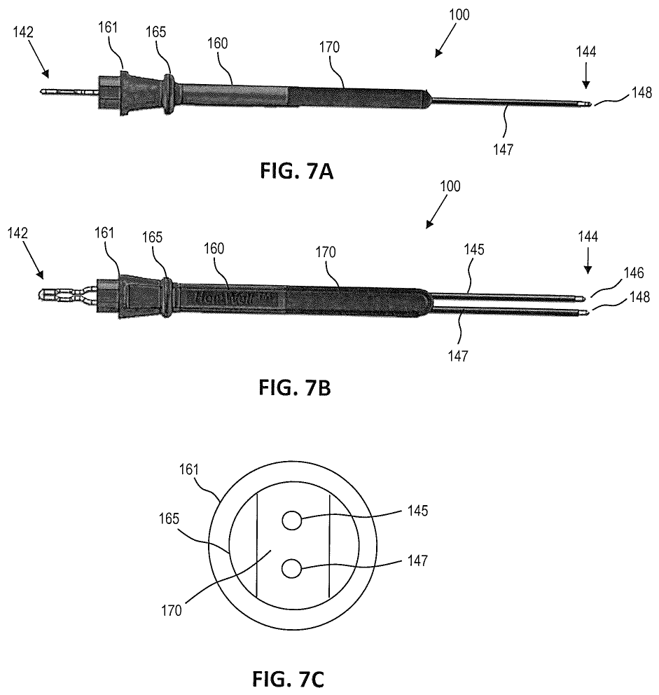

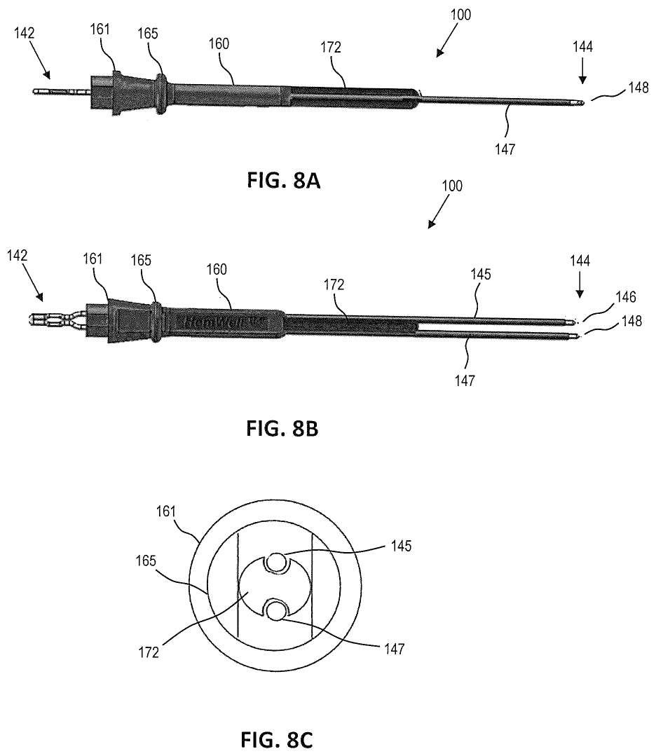

Referring now to FIGS. 7A-7C, the base overmold 160 may be formed with a closed extension 170. The closed extension 170 is a continuation of the base overmold 160 and partially extends over the elongated electrode members 145, 147. The closed extension 170 helps to ensure that the electrode tips 146 148 are held at the default spacing.

The base overmold 160 may be formed with base sections 161, 165 such that a portion of the base overmold 160 therebetween is tapered from base section 161 towards base section 165. From base section 165 towards the electrode tips 146, 148, a profile of the base overmold 160 matches a profile of the closed extension 170.

Referring now to FIGS. SA-8C, the base overmold 160 may be formed with an open extension 172. The open extension 172 is a continuation of the base overmold 160 but exposes the outward facing surfaces of the elongated electrode members 145, 147. The open extension 172 is formed between the inward facing surfaces of the elongated electrode members 145, 147. A profile of the open extension 172 is circular and includes a pair of openings to receive the inward facing surfaces of the elongated electrode members 145, 147 while the outward facing surfaces are exposed.

Although not shown, the final packaging of the probe electrode 100 includes a plastic guard to prevent accidental puncture and a gas permeable sleeve for sterilization. Due to the plastic being water-soluble, gas sterilization or E-Beam is used. Steam sterilization is prohibited.

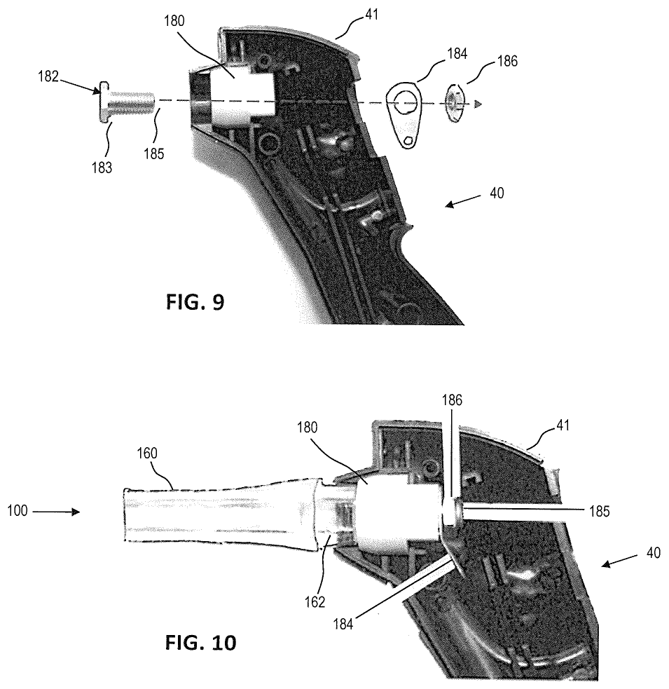

Referring now to FIGS. 9 and 10, a partially exploded view of the handheld device 40 configured to receive the probe electrode 100 will be discussed. The handheld device 40 includes a housing 41 that defines a shape of the handheld device 40. The housing 41 may be formed by joining together two housing halves. One of the housing halves 41 is removed to illustrate where the first end of the probe electrode 100 is to be coupled to the handheld device 40.

A plastic insert 180 is secured within the housing 41. The housing 41 includes a tapered extension and slotted fins within the housing. The plastic insert 180 is held in place by being positioned between the tapered extension and the slotted fins. The plastic insert 180 has an opening extending therethrough, and an electrical plug 182 is to be positioned within the opening. The electrical plug 182 has a receptacle end 183 and a threaded end 185. The receptacle end 183 is configured to receive the closed end 142 of the probe electrode 100. The threaded end 183 is to extend through a backside of the plastic insert 180.

An electrical lug 184 is to be positioned over the threaded end 185 of the electrical plug 182. An electrical wire will extend from the electrical lug 184 to the voltage output 42. The electrical wire provides a path for voltage from the voltage output 42 to the electrical plug 182 which is then transferred to the probe electrode 100. A nut 186 may be used to secure the electrical plug 182 and the electrical lug 184 to the plastic insert 180. The closed end 142 of the probe electrode 100 is inserted in the receptacle end 183 of the electrical plug 182, as shown in FIG. 10.

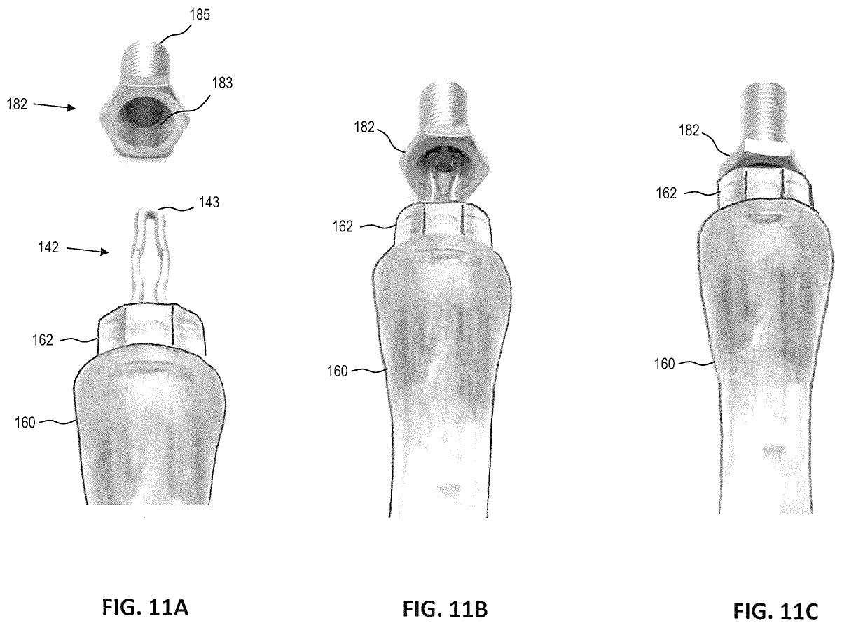

Referring now to FIGS. 11A-11C, a sequence of steps for inserting the closed end 142 of the probe electrode 100 into the receptacle end 183 of the electrical plug 182 will be discussed. As noted above, the electrical plug 182 is position within the opening in the plastic insert 180, and is secured to the plastic holder 180 using a nut 186 positioned on the threaded end 185 of the electrical plug 182

The closed end 142 of the probe electrode 100 is configured as a spring, as shown in FIG. 11A, for press-fitting into the receptacle end 183 of the electrical plug 182. As the tip 143 of the closed end 142 bottoms out within the receptacle end 183, the closed end 142 starts to expand outwards, as shown in FIG. 11B. With the closed end 142 fully inserted into the receptacle end 183 of the electrical plug 182, as shown in FIG. 11C, expansion of the closed end firmly holds the probe electrode 100 in place.

Although the plastic insert 180 is not shown, an outermost portion of the opening extending therethrough is shaped to receive the keying block 162 of the base overmold 160 of the probe electrode 100. Prior to insertion of the probe electrode 100, the keying block 162 may be rotated so that electrode tips 146, 148 are oriented in either a vertical or horizontal position with respect to how the electrode tips 146, 148 will be contacting the hemorrhoid 72.

Referring now to FIG. 12, the user interface membrane 190 positioned over the display area 53 of the handheld device 40 will be discussed. As noted above, the display 52 includes a current bar graph 54 and a timer 56. The user interface membrane 190 includes an opening for the current bar graph 54 and an opening for the timer 56.

The user interface membrane 190 is coupled to the controller 46 and provides user interface buttons for operating the handheld device 40. The user interface membrane 190 includes an on and up arrow button 192, an off and down arrow button 194, a self-test button 196 and a timer button 198.

A self-test of the handheld device 40 is to be run before each use, unless this function has been disabled by the doctor via software. To initiate the self-test, the doctor engages the grounding pad 80 with the probe electrode 100 and presses the self-test button 196.

During the self-test, each digit on the timer 56 is tested to make sure all the segments are working correctly. Then as part of the self-test, the handheld device 40 sweeps from delivering 0 to 16 milliamps (mA) and back to 0. As the current sweeps for 0 to 16 mA, the current bar graph 54 displays a bar at every 2 mA increment. When 16 mA is being delivered by the handheld device 40, then 8 bars appear. As the current sweeps back to 0, a bar will be removed every 2 mA decrement.

The self-test also requires the doctor to press the up arrow button 192 and the down arrow button 194 to make sure these buttons are working. If everything is working correctly, then “pass” is displayed on the timer 56. In order to remove display of “pass” the doctor presses the timer button 198. This causes all zeros to appear on the timer 56.

The doctor now has the option to operate the handheld device 40 in a manual mode or a timer mode. In the manual mode, the doctor engages the hemorrhoid 72 and then presses the up arrow button 192 for 3 seconds. The 3 second delay before current is delivered to the hemorrhoid 72 helps to prevent the handheld device 40 from accidentally delivering current when it is not intended to do so.

As current is being delivered to the hemorrhoid 72, the timer 56 counts up from zero. The handheld device 40 is configured to deliver 16 mA as a default current level setting. If the doctor wants less current to be delivered, then the doctor presses the down arrow button 194 until the desired number of bars appear on the timer 56, where each bar represents 2 mA.

If current is not being delivered to the hemorrhoid 72, then the timer 56 stops counting until current is actually being delivered again. For instance, the doctor may move the probe electrode 100 so that contact is no longer being made with the hemorrhoid 72. Once contact with the hemorrhoid 72 is again made by the probe electrode 100, then the timer 56 will display the bars indicating that current is being delivered by the handheld device 40. After an appropriate amount of time has passed in treating the hemorrhoid 72, the doctor presses the down arrow button 194.

For the doctor to operate the handheld device 40 in the timer mode, the doctor presses the timer button 198. This causes the timer 56 to flash or blink. Each press of the timer button 198 adds 30 seconds to the timer 56. After the desired amount of time has been entered, the doctor presses the timer button 56 again which causes the set time on the timer 56 to stop flashing. Next, the doctor presses the up arrow button 192 for 3 second. As current is being delivered to the hemorrhoid 72, the timer 56 counts down from the set time to zero.

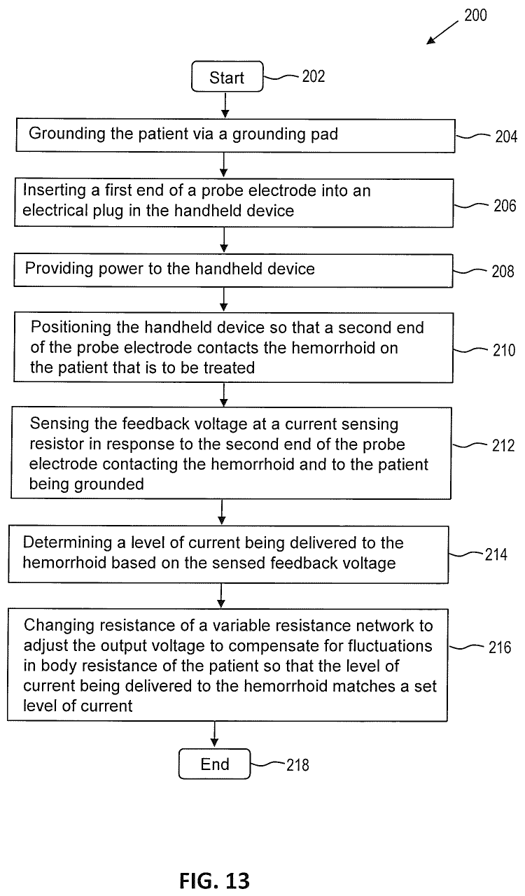

Another aspect is directed to a method for using the handheld device 40 as discussed above to treat a hemorrhoid 72 on a patient 70. Referring now to the flowchart 200 in FIG. 13, from the start (Block 202), the method includes grounding the patient 70 via the grounding pad 80 at Block 204. The first end of the probe electrode 100 is inserted into the electrical plug 182 at Block 206. Power is provided by the computing device 30 to the handheld device 40 over cable 60 at Block 208. The handheld device 40 is then positioned at Block 210 so that a second end of the probe electrode 100 contacts the hemorrhoid 72.

The controller 46 within the handheld device 40 is operated to sense the feedback voltage at the current sensing resistor 50 at Block 212 in response to the second end of the probe electrode 100 contacting the hemorrhoid 72 and to the patient 70 being grounded. A level of current being delivered to the hemorrhoid 72 based on the sensed feedback voltage is determined at Block 214.

The resistance of the variable resistance network 44 is changed at Block 216 to adjust the output voltage to compensate for fluctuations in body resistance of the patient 70 so that the level of current being delivered to the hemorrhoid matches a set level of current. The method ends at Block 218.

Yet another aspect is directed to monitoring usage of the handheld device 40 at a patient treatment center. The patient treatment center may also be referred to as a surgical center. As noted in the above Background section, a medical treatment handpiece is disclosed to treat hemorrhoids with the use of electrical current.

A limitation of the '966 patent is that usage of the medical treatment handpiece on each patient is self-reported by the patient treatment center. The patient treatment center provides a treatment summary report to a facility center. The facility center is responsible for providing the medical treatment handpiece to the patient treatment center, typically at no charge, and uses the treatment summary report to invoice or bill the patient treatment center for each patient treated.

The problem with self-reported usage of the medical treatment device by the patient treatment center is that the facility center is not able to verify accuracy of the treatment summary report. In addition, if the medical treatment handpiece is stolen, for example, and used elsewhere, then the facility center would not be aware of its usage. Consequently, there is a need to monitor usage of the above-described handheld device 40 to avoid the limitations associated with the medical treatment handpiece in the '966 patent.

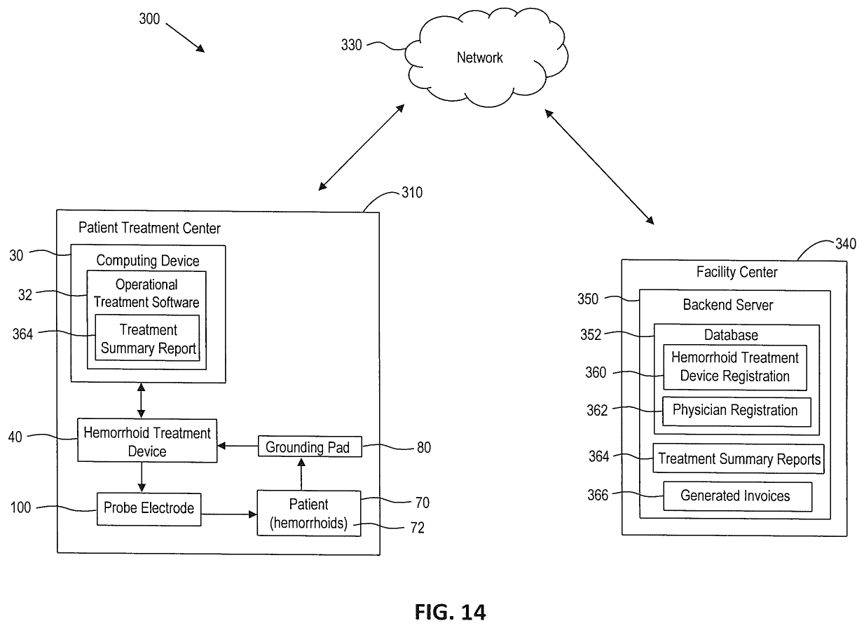

Referring now to FIG. 14, a hemorrhoid treatment system 300 will be discussed where usage of the handheld device 40 at a patient treatment center 310 is to be monitored. The handheld device 40 will also be referred to as a hemorrhoid treatment device 40.

The hemorrhoid treatment system 300 includes a backend server 350 at a facility center 340. The backend server 350 includes a database 352 for hemorrhoid treatment device registration 360 and physician registration 362. The hemorrhoid treatment device registration 360 keeps track of identification of the hemorrhoid treatment device 40 sent to the patient treatment center 310. The identification may be a serial number of the hemorrhoid treatment device 40, for example. The physician registration 362 keeps track of the physicians at the patient treatment center 310 authorized to use the hemorrhoid treatment device 40.

A computing device 30 at the patient treatment center 310 includes operational treatment software 32 that is configured to communicate with the backend server 350 via a network 330. The network 330 provides internet access and may be configured in any combination of wired and wireless networks.

An administrator at the patient treatment center 310 first registers the hemorrhoid treatment device 40 at the backend server 350. Once registered, then the operational treatment software 32 is downloaded over the network 330 to the computing device 30. At this time, the administrator is also able to register the physicians at the patient treatment center 310 authorized to use the hemorrhoid treatment device 40.

The computing device 30 is configured to provide power to the hemorrhoid treatment device 40 over a USB connection with the hemorrhoid treatment device 40. The operational treatment software 32 communicates with the backend server 350 in response to the hemorrhoid treatment device 40 being registered. In response to the registered hemorrhoid treatment device 40 being plugged into the computing device 40, then a self-test is to be performed on the hemorrhoid treatment device 40 before the physician logs into the operational treatment software 32. After a successful self-test has been performed and the physician logs into the operational treatment software 32, then the hemorrhoid treatment device 40 is operational for use.

In response to the hemorrhoid treatment device 40 being operational, an output voltage is delivered to the probe electrode 100 coupled to the hemorrhoid treatment device 40, with the probe electrode 100 to contact the hemorrhoid 72 being treated. The output voltage is adjusted to compensate for fluctuations in body resistance of the patient 70 so that a level of current being delivered to the hemorrhoid 72 matches a set level of current. The probe electrode 100 is a single conductor that delivers a monopolar direct current to the hemorrhoid 72 where a return path is provided via the grounding pad 80.

The hemorrhoid treatment device 40 includes a voltage output 42 configured to deliver the output voltage to the probe electrode 100, and a variable resistance network 44 coupled to the voltage output 42, as shown in FIG. 1. Operating the hemorrhoid treatment device 40 includes changing resistance of the variable resistance network 44 to adjust the output voltage to compensate for the fluctuations in body resistance of the patient 70 so that the set level of current being delivered to the hemorrhoid is consistent. The grounding pad 80 is positioned to contact the patient 70, and is used in determining the fluctuations in body resistance of the patient 70.

The operational treatment software 32 is configured to monitor usage of the hemorrhoid treatment device 40 by the physician to treat a patient 70 with hemorrhoids 72. The operational treatment software 32 is further configured to generate a generate a treatment summary report 364 in real-time as the patient is being treated. After completion of the treatment to the patient 70, the treatment summary report 364 is then transmitted to the backend server 350 via the network 330.

The treatment summary report 364 includes identification of the patient, how many hemorrhoids were treated, and how long current was delivered to the hemorrhoid 72 by the hemorrhoid treatment device 40. In response to receiving the treatment summary report 364, the backend server 350 generates an invoice 366 that is to be sent to the patient treatment center 310.

As noted above, an administrator at the patient treatment center 310 registers the hemorrhoid treatment device 40 at the backend server 350. This is done via an administrative portal. The administrative portal also allows the administrator to download the operational treatment software 32 on the computing device 30, and to register the physicians at the patient treatment center 310 authorized to use the hemorrhoid treatment device 40.

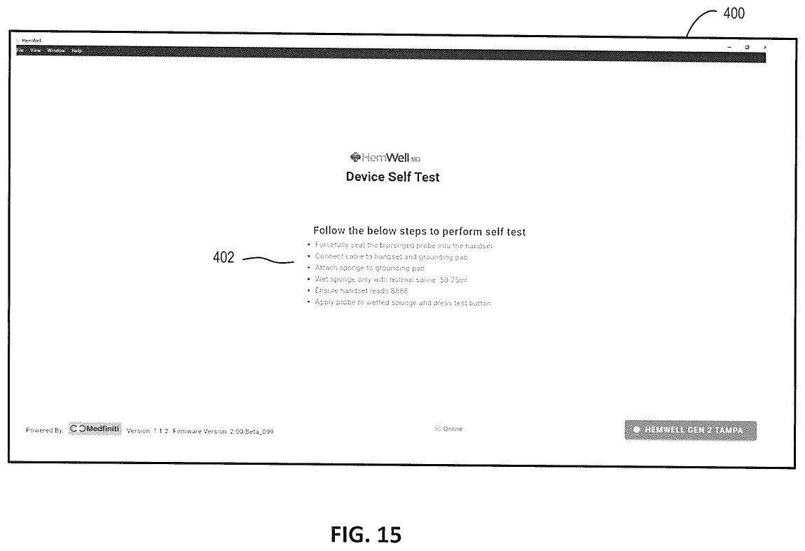

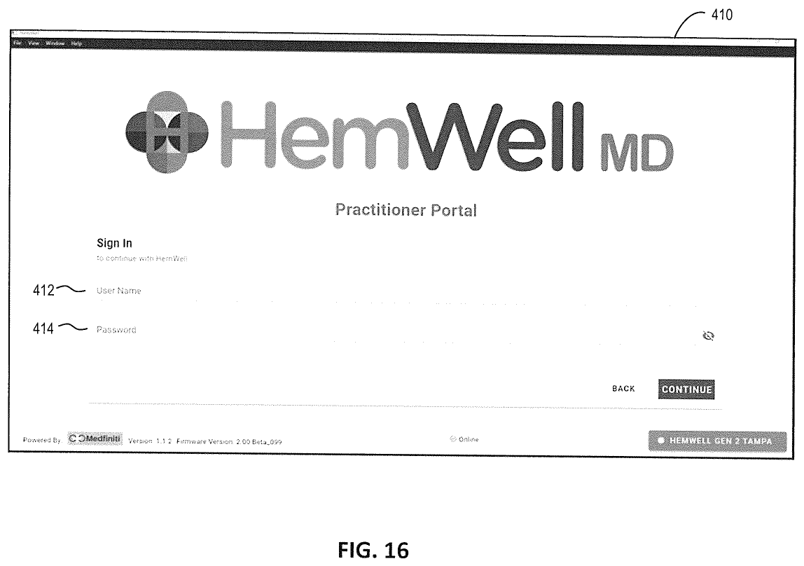

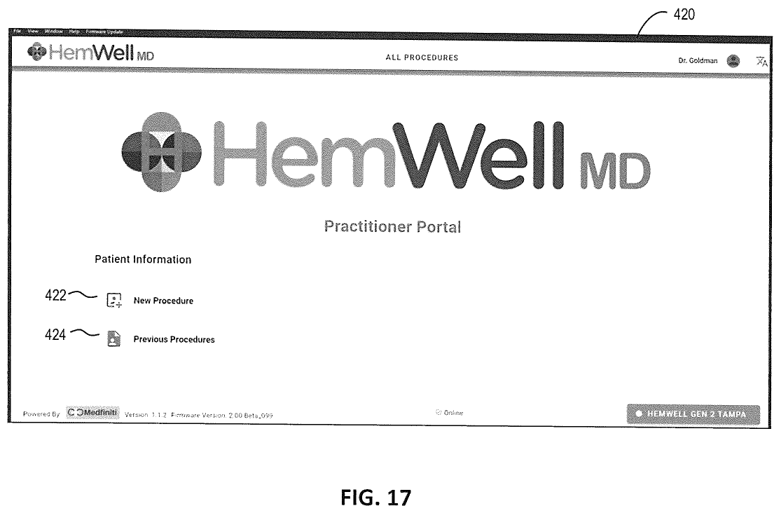

Once the operational treatment software 32 has been downloaded on the computing device 30, and the hemorrhoid treatment device 40 and the physicians at the patient treatment center 310 have been registered with the backend server 340, then the physicians interface with the operational treatment software 32 via a practitioner portal, which will now be discussed in reference to the screenshots in FIGS. 15-24.

Initially, before the physician logs into the operational treatment software 32, the hemorrhoid treatment device 40 is plugged into the computing device 30, and is to undergo a self-test before use, as provided in screenshot 400 shown in FIG. 15. To initiate the self-test, the physician follows the steps provided in section 402. Once the steps have been completed, then the physician presses the self-test button 196 displayed on the user interface membrane 190, as shown in FIG. 12. After a successful self-test, the hemorrhoid treatment device 40 may be used on multiple patients during the day without having to repeat the self-test.

Screenshot 410 is displayed after the successful self-test, as shown in FIG. 16. The physician logs into the operational treatment software 32 by entering a username in section 412 and a password in section 414. The operational treatment software 32 allows the backend server 350 to verify that the physician is registered to use the hemorrhoid treatment device 40.

After registration of the physician has been verified by the backend server 350, screenshot 420 is displayed, as shown in FIG. 17. This allows the physician to initiate a new procedure as provided in section 422, or to return to a previous procedure in section 424.

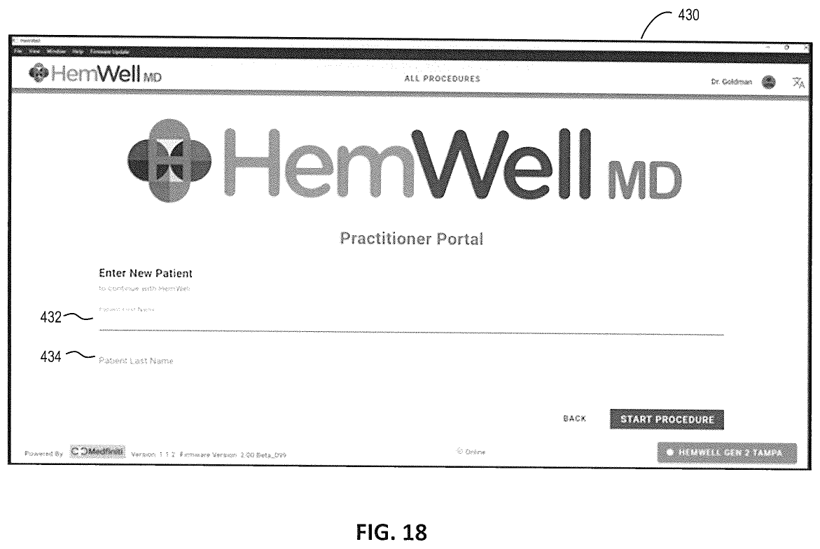

In response to the physician selecting a new patient in section 422, screenshot 430 is displayed, as shown in FIG. 18. The physician enters the patient's first name in section 432 and last name in section 434.

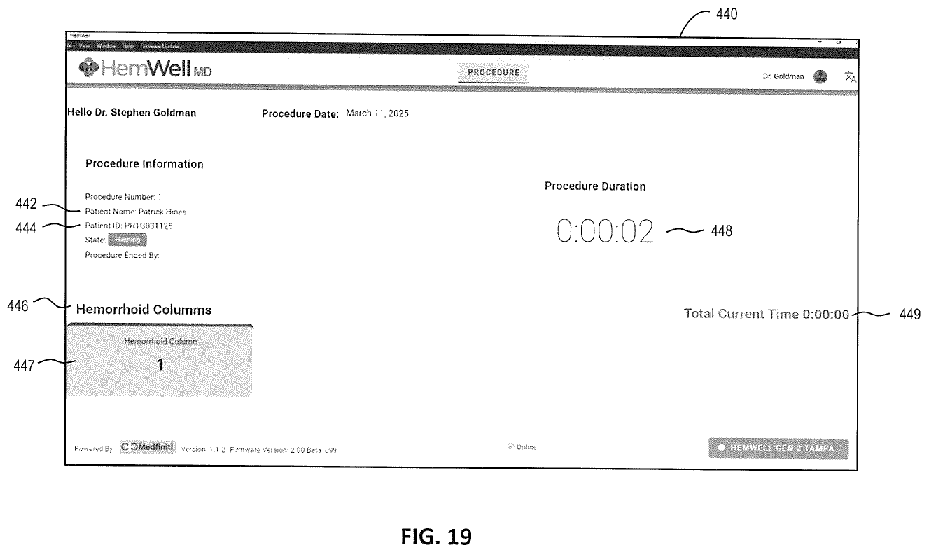

Screenshot 440 as shown in FIG. 19 is displayed at the start of the procedure. To comply with Health Insurance Portability and Accountability Act (HIPPA) rules, a patient ID is assigned to the patient in section 444 under the patient's name in section 442. When the treatment summary report 364 is sent to the backend server 350, the treatment summary report 364 includes the patient's ID 444 but not the patient's name 442.

The hemorrhoid columns in section 446 keeps track of the number of hemorrhoids 72 being treated on the patient 70. The first hemorrhoid 72 being treated is reflected in section 447 which is labeled as hemorrhoid column 1. The overall procedure duration using the hemorrhoid treatment device 40 is provided in section 448, and the total current time being applied to the hemorrhoids 72 being treated is provided in section 449. Billing is based on total current time 449 and not the overall procedure duration 448. Since the displayed total current time is 0, no current has been applied to the hemorrhoid 72. That is, the probe electrode 100 is not in contact with the hemorrhoid 72. The color of section 447 may be yellow, for example, indicating that current is not flowing through the hemorrhoid 72.

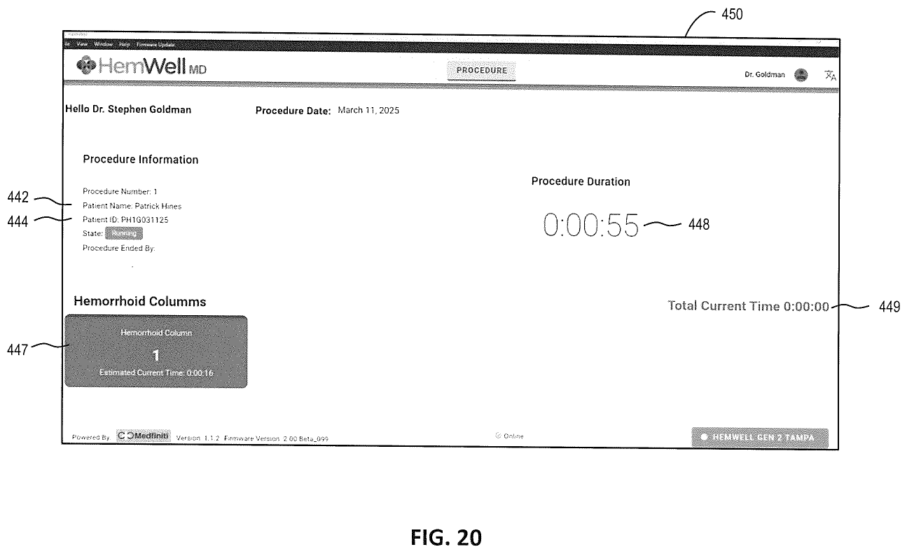

In screenshot 450, as shown in FIG. 20, current is being applied to the hemorrhoid 72. The color of section 447 may now be red, for example, indicating that current is flowing through the hemorrhoid 72. In section 447 an estimated current time of 16 seconds has been applied to the hemorrhoid 72. However, the total current time in section 449 will not show or display the total current time until the hemorrhoid 72 has been treated.

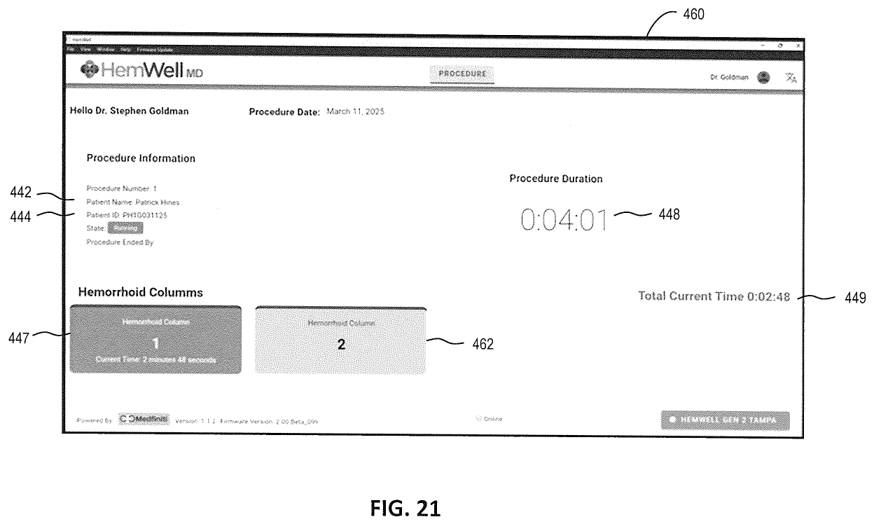

In screenshot 460, as shown in FIG. 21, the color of section 447 for the first hemorrhoid 72 may now be blue, for example, indicating that the first hemorrhoid 72 has been treated. Now the total current time of 2:48 is displayed in the total current time section 449. A second hemorrhoid 72 is shown as being treated next. The second hemorrhoid 72 is reflected in section 462 which is labeled as hemorrhoid column 2. The color of section 447 for the first hemorrhoid 72 may now be blue, for example, indicating that the first hemorrhoid 72 has been treated. Since current has not been applied to the second hemorrhoid 72, the color of section 462 is yellow. In this case, the total current time in section 449 corresponds to the duration of current applied to the first hemorrhoid 72.

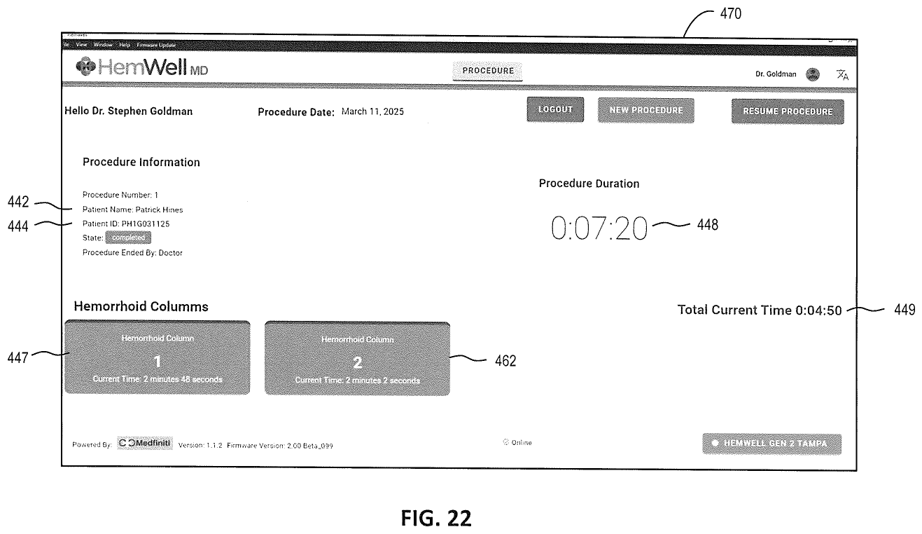

After the second hemorrhoid 72 has been treated, the color of section 462 is now blue, as provided in screenshot 470 shown in FIG. 22. The current time in section 447 for the first hemorrhoid 72 and the current time in section 462 for the second hemorrhoid 72, when added together, equals the total current time in section 449. The total current time in section 449 is less than the overall procedure duration in section 448.

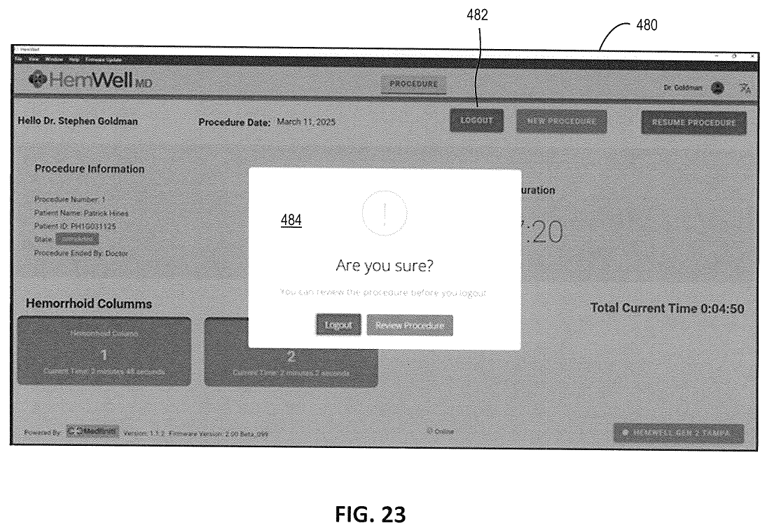

After treatment of the patient 70 has been completed, then the physician may log out by selecting section 482, as provided in screenshot 480 shown in FIG. 23. Section 484 prompts the physician for confirmation that the procedure has been completed.

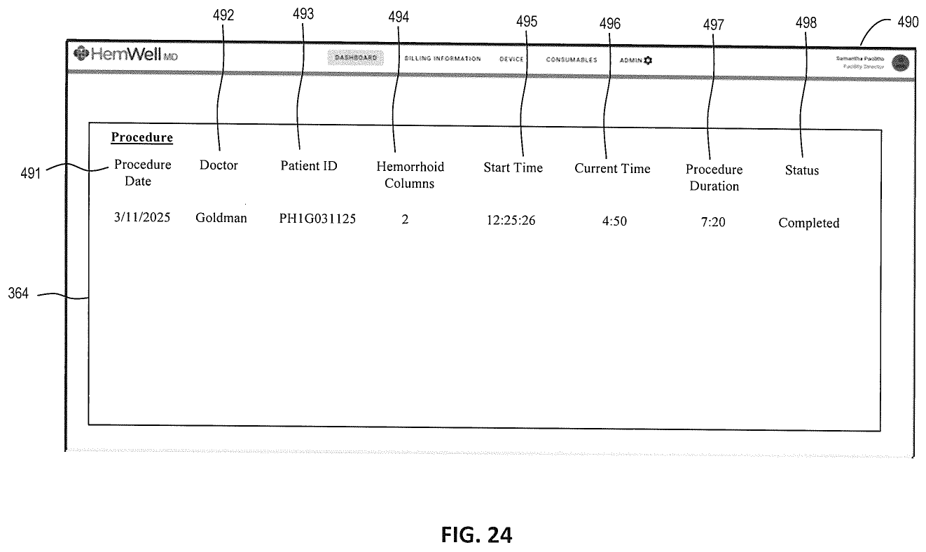

The operational treatment software 32 is configured to generate a treatment summary report 364 in real-time after completing treatment of the patent 70, as provided in screenshot 490 shown in FIG. 24. The treatment summary report includes the following: date of the procedure in column 491, name of the doctor in column 492, patient ID in column 493, number of hemorrhoids treated in column 494, start time in column 495, current time in column 496, procedure duration in column 491 and a status of the procedure in column 498. The backend server receives the treatment summary report 364, and is able to timely generate an invoice 366 for the patient treatment center 310. If the patient treatment center 310 fails to pay the invoice, then the hemorrhoid treatment device 40 may be disabled by the backend server 350 at the facility center 340.

Referring now to the flowchart 600 in FIG. 24, a method for monitoring usage of the hemorrhoid treatment device 40 will be discussed. From the start (Block 602), the method includes registering with the backend server 350 the hemorrhoid treatment device 40 provided to the patient treatment center 310 at Block 604. A physician at the patient treatment center 310 is registered at Block 606 with the backend server 310 to operate the hemorrhoid treatment device 40.

The hemorrhoid treatment device 40 is connected to the computing device at Block 608. The computing device 40 includes operational treatment software 32 configured to communicate with the backend server 350. The physician logs into the operational treatment software 32 on the computing device 40 at Block 610. The operational treatment software 32 verifies with the backend server 350 that the physician is registered to operate the hemorrhoid treatment device 40.

The hemorrhoid treatment device 40 is operated by the physician at Block 612 after verification of the physician by the backend server 350. The hemorrhoid treatment device is operated to treat the patient 70 with hemorrhoids 72. Usage of the hemorrhoid treatment device 40 by the physician is monitored at Block 614 via the operational treatment software 32.

A treatment summary report 364 is generated at Block 616 via the operational treatment software 32 after treatment of the patient 70 with hemorrhoids 72 has been completed. The treatment summary report 364 is provided to the backend server 350 at Block 618 via the operational treatment software 32. An invoice is generated 366 at Block 620 in response to the backend server 350 receiving the treatment summary report 364. The method ends at Block 622.

Yet another aspect is directed to operating the hemorrhoid treatment device 40 while the patient 70 is under general anesthesia or conscious sedation. This advantageously allows the maximum allowable current level to be delivered to every patient 70 when treating hemorrhoids 72 without requiring patient feedback while the hemorrhoids 72 are being treated. Currently, the Food and Drug Administration (FDA) has determined that this level is 16 milliamps (mA).

Typically, only a small percentage of individuals can tolerate a maximum current level of 8 mA without being under general anesthesia or conscious sedation. A limitation of the '966 patent is that the patient is awake. The current level starts low (2 mA) to prevent shocking the patient, and over the course of a few minutes (10 minutes) the current level increases to the patient's pain limit. At this point the current is decreased by 2 mA to below the threshold of marked pain to the threshold of tolerable pain. A further limitation of this approach is that the patient 70 would have to return for several treatments to fully treat the hemorrhoids 72.

In contrast, the success rate in treating hemorrhoids 72 at the maximum current level while the patient 70 is under general anesthesia or conscious sedation is significantly increased to 90% or higher. When the patient 70 is under general anesthesia or conscious sedation, the maximum current level may be instantly applied to the patient 70. This significantly contributes to the success rate in treating hemorrhoids 72. That is, the set maximum current level may be applied in 5 seconds are less, for example. The maximum current level may typically be within a range of 12-16 mA, for example. However, if the FDA increases the maximum allowed current level, then the hemorrhoid treatment device 40 may be configured to deliver more than 16 mA.

The use of general anesthesia allows for pain-free surgical procedures without requiring patient 70 feedback during the procedure. General anesthesia is a medically induced state of unconsciousness, analgesia (loss of pain), and muscle relaxation that allows for surgical procedures to be performed without the patient's awareness or discomfort.

The use of conscious sedation is a safe and effective option for patients who need to undergo medical procedures while maintaining a level of consciousness. Conscious sedation is a medical procedure that involves administering medications to the patient to induce sleep. The patient is still able to breath on their own but is unconscious, and does not respond to verbal or painful stimuli. Feedback from the patient 70 is not needed during the procedure. Conscious sedation provides a relaxed and comfortable experience while minimizing the risks associated with general anesthesia.

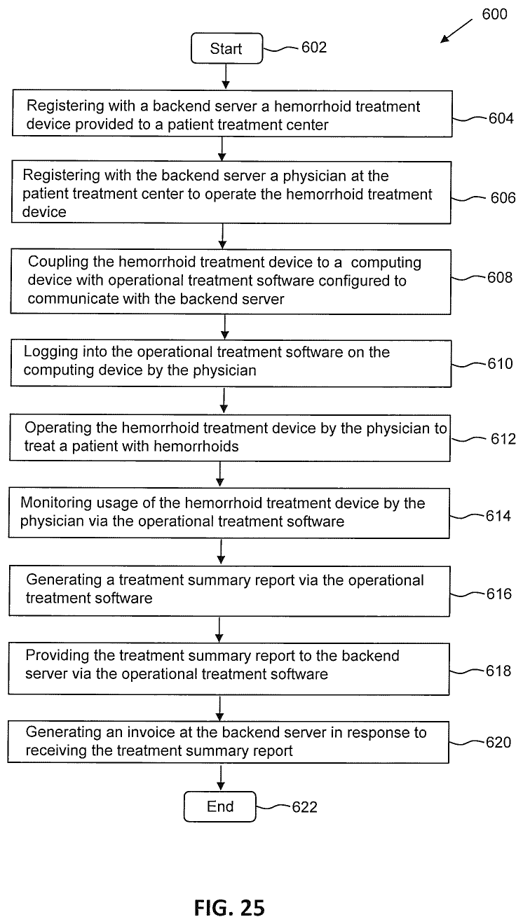

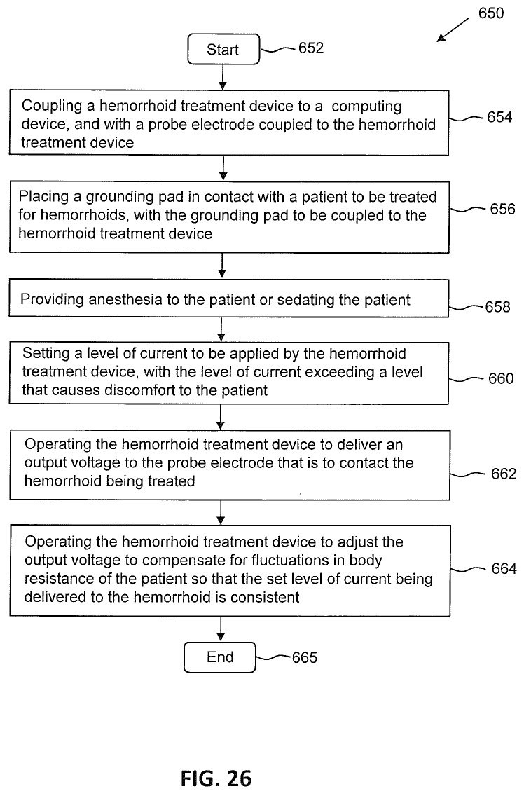

Referring now to the flowchart 650 in FIG. 25, a method for operating the hemorrhoid treatment device 40 will be discussed. From the start (Block 652), the method includes coupling the hemorrhoid treatment device 40 to the computing device 30 with a cable 60 at Block 654. The probe electrode 100 is coupled to the hemorrhoid treatment 40.

A grounding pad 80 is placed in contact with the patient 70 to be treated for hemorrhoids 72 at Block 656. The grounding pad 80 is coupled to the hemorrhoid treatment device 40. The patient 70 is placed under general anesthesia or conscious sedation at Block 658. A level of current to be applied by the hemorrhoid treatment device 40 is set at Block 660, with the level of current exceeding a level that causes discomfort to the patient 70.

The hemorrhoid treatment device 40 is operated at Block 662 to deliver an output voltage to the probe electrode 100 coupled to the hemorrhoid treatment device 40, with the probe electrode 100 to contact the hemorrhoid 72 being treated. The probe electrode 100 is a single conductor that delivers a monopolar direct current to the hemorrhoid 72 where a return path is provided via the grounding pad 80. The hemorrhoid treatment device 40 is further operated at Block 664 to adjust the output voltage to compensate for fluctuations in body resistance of the patient 70 so that the set level of current being delivered to the hemorrhoid is consistent. The method ends at Block 665.

As discussed above, the hemorrhoid treatment device 40 includes a voltage output 42 configured to deliver the output voltage to the probe electrode 100, and a variable resistance network 44 coupled to the voltage output 42. The hemorrhoid treatment device 40 is operated to change resistance of the variable resistance network 44 to adjust the output voltage to compensate for the fluctuations in body resistance of the patient 70 so that the set level of current is consistently delivered to the hemorrhoid. The set level of current may be within a range of 12-16 mA. Alternatively, the set level of current may be equal to or greater than 16 mA.

The fluctuations in body resistance of the patient 70 is based on an interface between the grounding pad 80 and the patient 70. The cable 60 coupling the hemorrhoid treatment device 40 to the computing device 30 includes an inline connector junction 62. The grounding pad 80 is coupled to the hemorrhoid treatment device 40 via the inline connector junction 62.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A method for monitoring usage of a hemorrhoid treatment device comprising:
registering with a backend server the hemorrhoid treatment device provided to a patient treatment center;
registering with the backend server a physician at the patient treatment center to operate the hemorrhoid treatment device;
coupling the hemorrhoid treatment device to a computing device at the patient treatment center, with the computing device including operational treatment software configured to communicate with the backend server;
logging into the operational treatment software on the computing device by the physician, with the operational treatment software verifying with the backend server that the physician is registered to operate the hemorrhoid treatment device;
operating the hemorrhoid treatment device by the physician to treat a patient with hemorrhoids;
monitoring usage of the hemorrhoid treatment device by the physician via the operational treatment software; and
generating a treatment summary report via the operational treatment software after the patient with hemorrhoids has been treated.

2. The method according to claim 1 comprising:
providing the treatment summary report to the backend server.

3. The method according to claim 2 wherein the backend server generates an invoice for the patient treatment center in response to receiving the patient treatment report.

4. The method according to claim 3 wherein the backend server sends the invoice to the patient treatment center.

5. The method according to claim 1 comprising:
performing a self-test on the hemorrhoid treatment device before the physician logs into the operational treatment software.

6. The method according to claim 1 wherein the treatment summary report includes identification of the patient, how many hemorrhoids were treated, and how long current was delivered to the hemorrhoids by the hemorrhoid treatment device.

7. The method according to claim 1 wherein the hemorrhoid treatment device is powered by the computing device, and a probe electrode is coupled to the hemorrhoid treatment device to deliver a monopolar direct current to the hemorrhoids being treated on the patient.

8. The method according to claim 1 wherein operating the hemorrhoid treatment device comprises:
delivering an output voltage to a probe electrode coupled to the hemorrhoid treatment device, with the probe electrode to contact the hemorrhoid being treated; and
adjusting the output voltage to compensate for fluctuations in body resistance of the patient so that a level of current being delivered to the hemorrhoid matches a set level of current.

9. The method according to claim 8 wherein the hemorrhoid treatment device comprises a voltage output configured to deliver the output voltage to the probe electrode, and a variable resistance network coupled to the voltage output, and operating the hemorrhoid treatment device comprises:
changing resistance of the variable resistance network to adjust the output voltage to compensate for the fluctuations in body resistance of the patient so that the set level of current being delivered to the hemorrhoid is consistent.

10. The method according to claim 8 comprising:
positioning a grounding pad to contact the patient, with the grounding pad being coupled to the hemorrhoid treatment device to be used in determining the fluctuations in body resistance of the patient.

11. A hemorrhoid treatment system comprising:
a backend server;
a computing device at a patient treatment center, with the computing device including operational treatment software in communications with the backend server; and
a hemorrhoid treatment coupled to the computing device, with the hemorrhoid treatment device being registered with the backend server, with the hemorrhoid treatment device to be operated by a physician at the patient treatment center after logging into the operational treatment software, with the physician being registered with the backend server to operate the hemorrhoid treatment device, and with the operational treatment software being configured to perform the following:
monitor usage of the hemorrhoid treatment device by the physician to treat a patient with hemorrhoids via the operational treatment software, and
generate a treatment summary report via the operational treatment software after the patient with hemorrhoids has been treated.

12. The hemorrhoid treatment system according to claim 11 wherein the operational treatment software is configured to provide the treatment summary report to the backend server.

13. The hemorrhoid treatment system according to claim 12 wherein the backend server is configured to generate an invoice for the patient treatment center in response to receiving the patient treatment report.

14. The hemorrhoid treatment system according to claim 13 wherein the backend server is configured to send the invoice to the patient treatment center.

15. The hemorrhoid treatment system according to claim 11 wherein the hemorrhoid treatment system is operated to perform a self-test before the physician logs into the operational treatment software.

16. The hemorrhoid treatment system according to claim 11 wherein the treatment summary report includes identification of the patient, how many hemorrhoids were treated, and how long current was delivered to the hemorrhoids by the hemorrhoid treatment device.

17. The hemorrhoid treatment system according to claim 11 wherein the hemorrhoid treatment device is powered by the computing device, and a probe electrode is coupled to the hemorrhoid treatment device to deliver a monopolar direct current to the hemorrhoids being treated on the patient.

18. The hemorrhoid treatment system according to claim 11 wherein the hemorrhoid treatment device is configured to perform the following:
deliver an output voltage to a probe electrode coupled to the hemorrhoid treatment device, with the probe electrode to contact the hemorrhoid being treated; and
adjust the output voltage to compensate for fluctuations in body resistance of the patient so that a level of current being delivered to the hemorrhoid matches a set level of current.

19. The hemorrhoid treatment system according to claim 18 wherein the hemorrhoid treatment device comprises:
a voltage output configured to deliver the output voltage to the probe electrode;
a variable resistance network coupled to the voltage output; and
a controller coupled to the variable resistance network and configured to change resistance of the variable resistance network to adjust the output voltage to compensate for the fluctuations in body resistance of the patient so that the set level of current being delivered to the hemorrhoid is consistent.

20. The hemorrhoid treatment system according to claim 18 comprising:
a grounding pad positioned to contact the patient, with the grounding pad to be coupled to the hemorrhoid treatment device to be used in determining the fluctuations in body resistance of the patient.
